(19) United States
(12) Patent Application Publication
(10) Pub. No.: US 2004/0210275 A1
(43) Pub. Date: Oct. 21, 2004

(54) Method of stimulating collagen formation

(76) Inventors: Godfrey Arthur Town, West Sussex (GB); John Colles, Lanarkshire (GB)

Correspondence Address: Gregory J Lavorgna, Drinker Biddle & Reath, One Logan Square, 18th & Cherry Streets, Philadelphia, PA, 19103-6996, US

(21) Appl. No.: 10/477,503

(22) PCT Filed: May 8, 2002
(86) PCT No.: PCT/EP02/05082

(30) Foreign Application Priority Data
May 9, 2001 (GB) 01111271.3

(51) Int. Cl. A61N 5/06

(52) U.S. Cl. 607/88

(57) ABSTRACT

A method of stimulating collagen formation in a selected area of mammalian skin in which the selected area is irradiated with a source of visible or near infra-red radiation. The radiation is absorbable by intracellular chromophores to enhance cellular activity and increase collagen formation. A skin treatment device for carrying out the method of the invention is also described.

### BACKGROUND TO THE INVENTION

This invention relates to a method of stimulating collagen formation in mammalian skin.

The application of lasers to cosmetic improvement of skin tissue is well known. Historically the carbon dioxide laser was first used to ablate thin layers of epidermal tissue with the consequent repair mechanisms inducing new collagen formation and a reduction in the, for example, depth of wrinkles. This process is equivalent to surgical removal of the outermost layers but includes a significant amount of residual tissue heating which is thought to contribute to the overall process. Similarly, the mechanical process of dermabrasion has also been employed in the prior art.

More recently the erbium:YAG laser has been used for the same process. In this case, however, the significantly smaller absorption depth encountered at this laser's wavelength of 3 μm enables thinner layers to be removed than with the CO₂ laser. This allows for a less invasive process but nevertheless still one that requires epidermal removal.

A number of new approaches have been introduced with the specific goal of removing wrinkles from superficial areas of skin without the prior removal of the epidermis thus providing less invasive techniques. These techniques involve absorbing laser or other light source radiation in chromophores within the dermal layers and utilising the resulting temperature rises in the surrounding tissue to stimulate collagen reformation. The chromophores as taught by U.S. Pat. No. 5,983,900 are either naturally occurring such as haemoglobin or, potentially, an artificial chromophore which, when applied topically in an appropriate solution, could diffuse to the dermal region.

The choice of laser wavelength or light source band of wavelengths is dictated by the absorption characteristics of the chosen chromophore thus, in the case of haemoglobin, a dye laser tuned to operate at an absorbing wavelength of oxy-haemoglobin would be an appropriate choice. Others working in the same field propose using absorption into the water contained within skin and thus advocate the use of laser or light source wavelengths in the near and medium infra-red (e.g. around 1000 nm to 2000 nm). The effectiveness of any of these techniques remains not fully proven.

Indeed Clement teaches that it is still sometimes desirable to remove part of the epidermis prior to irradiating the dermal layer as described above. Additionally Clement teaches a preferred choice of pulse duration for the irradiation in the 100 μs to 1 ms range. This is consistent with the physical principles of selective photothermolysis first introduced to the field by Anderson, R. R. and Parrish, J. A. “Selective Photothermolysis”; Science 1983 Vol 220 pp 524-527 which teaches that a match between the thermal characteristics of the absorbing species and the pulse duration enhances the localised heating of the target and reduces the degree of generalised thermal damage to the surrounding tissue.

The methods of the prior art therefore employ thermal interactions to promote the stimulation of collagen growth with the resultant potential disadvantages of epidermal removal and/or skin damage.

An object of the invention is to overcome the problems of the prior art.

### SUMMARY OF THE INVENTION

According to the invention there is provided a method of stimulating collagen formation in mammalian skin comprising irradiating the skin with radiation wherein the radiation is absorbable by intracellular chromophores to enhance cellular activity and increase collagen formation.

Preferably, the intracellular chromophores comprise cytochromes. More preferably, the intracellular chromophores comprise mitochondrial cytochromes.

Suitably, the enhanced cellular activity comprises an increase in pro-collagen formation to subsequently increase collagen formation.

Suitably, the enhanced cellular activity comprises photo mechanical or photo chemical activity.

Advantageously, the radiation comprises visible or near infra-red radiation.

Suitably, the irradiation is provided by an incoherent source suitably filtered to provide the visible or near infra-red radiation. Preferably, the incoherent source or radiation source comprises a laser source. Suitably, the laser comprises a copper bromide laser.

Preferably, the delivered energy density of the radiation to the skin is between 0.5 and 30 J/cm². More preferably, the energy density is between 2 and 15 J/cm².

Advantageously, the irradiation source comprises a pulsed output. Suitably, the pulse duration is between 1 ns and 1 ms. More preferably, the pulse duration is between 1 ns and 500 ns.

Preferably, the pulse comprises a sharp leading edge.

In a preferred embodiment of the invention, the radiation has a wave length of about 578 nm.

The invention also extends to the use of visible or near infra-red radiation absorbable by intracellular chromophores in the treatment of mammalian skin to reduce skin imperfections such as wrinkles, rough textures or other blemishes from a selected area. Preferably, the radiation is absorbable by a cytochrome. More preferably, the cytochrome comprises a mitochondrial cytochrome.

The invention also extends to a method of reducing skin imperfections such as wrinkles, rough textures or other blemishes from a selected area of mammalian skin comprising stimulating collagen formation in the skin as hereinbefore defined.

In a further embodiment, the invention extends to a skin treatment device for stimulating collagen formation in mammalian skin comprising a radiation source absorbable by intracellular chromophores and an applicator communicable with the radiation source for applying the radiation to mammalian skin. Preferably, the radiation source comprises radiation absorbable by cytochromes.

More preferably, the radiation comprises radiation absorbable by mitochondrial cytochromes.

Suitably, the radiation is provided by an incoherent source suitably filtered to provide visible or near infra-red light.

Advantageously, the radiation source comprises a laser and preferably the laser comprises a copper bromide laser. More preferably, the laser comprises a dye laser tuned to a wavelength absorbable by the mitochondrial chromophores.

Suitably, the applicator is communicable with the radiation source via a fibre or articulating optical device.

Advantageously, the applicator comprises a hand piece. Alternatively, the applicator comprises a scanning device for the controlled irradiation of an enlarged skin area.

The present invention therefore relates to the stimulation of pro-collagen, the subsequent formation of new collagen and thus remodelling of sub-epidermal collagen in mammalian skin tissue. Such remodelling is a factor in the reduction of and in some cases removal of wrinkles and similar age and environment related attributes of human skin. Other malformations such as the stretch marks associated with post birth contraction of tissue and scars associated with injury or skin diseases such as acne could similarly benefit.

The invention provides both a method and apparatus which achieve wrinkle removal through the use of either one or both of photomechanical and photochemical means. The method and apparatus of the invention provide a minimally invasive technique by which skin geometry may be altered and utilises laser or light source parameters less likely to cause unwanted residual tissue damage.

The invention will now be described having regard to the following non-limiting examples and drawings in which:

### DESCRIPTIONS OF THE DRAWINGS

FIG. 1 is a schematic cross-sectional view of human skin showing the structural composition of the epidermis and the dermis;

FIG. 2 is a schematic representation of the biochemical sequences involved in the formation of collagen fibres, and

FIG. 3 is a schematic diagram of a skin treatment device according to the present invention in use.

### DETAILED DESCRIPTION OF THE INVENTION

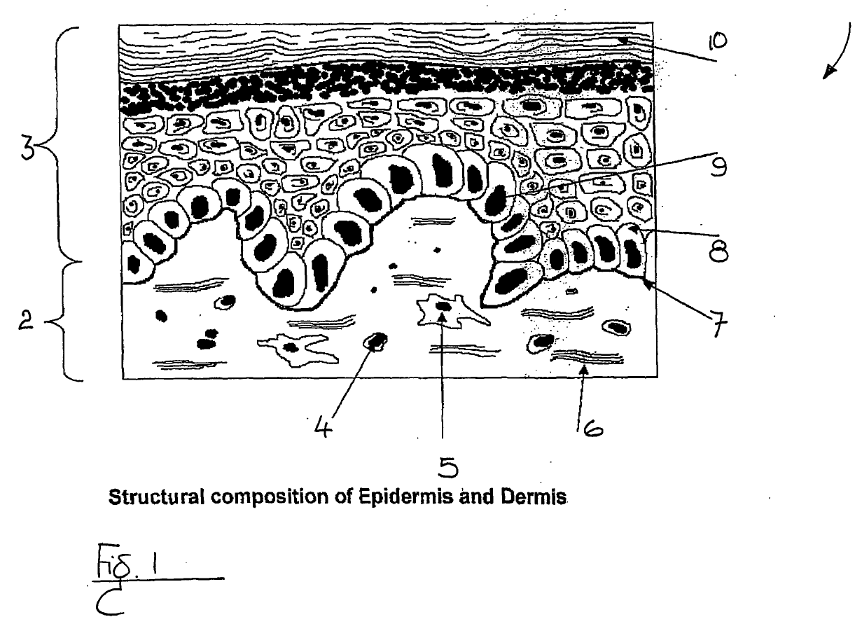

FIG. 1 is a schematic cross-sectional view of a portion of human skin 1 which should be familiar to those skilled in the art. The skin 1 is made up, generally, of an inner dermis 2 and an outer epidermis 3. The dermis 2 is provided with blood vessels 4, fibroblasts 5 and collagen fibres 6.

The dermis 2 is separated from the epidermis 3 at a basement membrane 7. Basal cells 8 are located at the basement membrane 7 and are provided with melanoctyes 9.

Finally, the outer surface of the epidermis 3 is provided with a stratum corneum 10 which provides a protective layer on the epidermis 3.

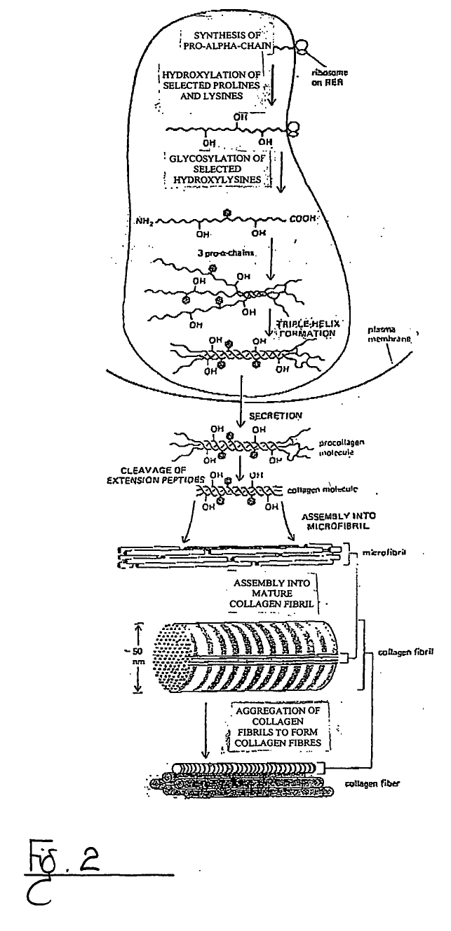

FIG. 2 is a schematic representation of the synthesis of collagen fibres from pro-alpha chains in a cell. As shown in the drawing, following the synthesis of pro-alpha chains, selected prolines and lysines are hydroxylated followed by glycosylation of selected hydroxylysines to result in the ultimate formation of a triple-helix.

Following secretion through a plasma membrane, a procollagen molecule is formed which is then converted into a collagen molecule. The collagen molecule is assembled into a microfibril which is in turn ultimately assembled into a mature collagen fibril.

Collagen fibrils are ultimately aggregated to form collagen fibres.

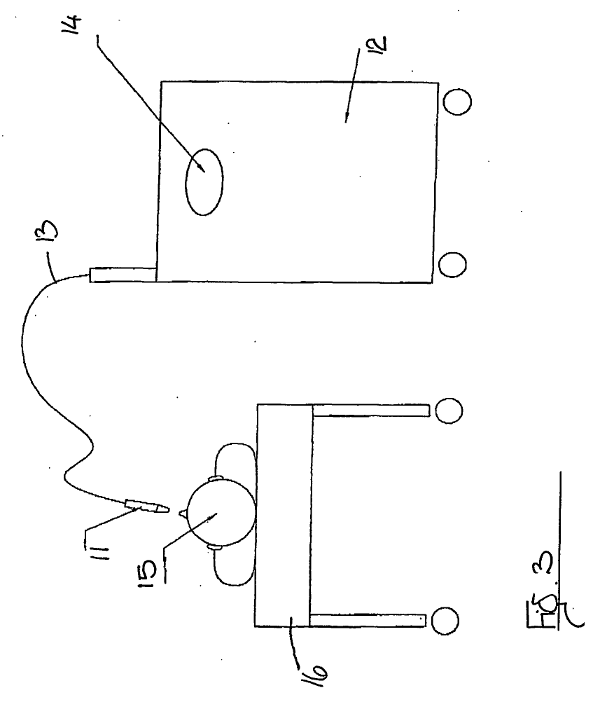

FIG. 3 is a schematic diagram of a skin treatment device in accordance with the invention in use. The device of the invention is provided with a hand piece 11 connected to a housing 12 containing a radiation source (not shown) by an optical cable 13 comprising a bundle of optical fibres. The radiation source is typically a laser providing visible or near infra-red light and is controllable by means of a control panel 14 on the housing 12. A patient 15 lies on a bed 16 and the hand piece 11 is held by an operator (not shown). The hand piece 11 is relatively light and can therefore be moved relatively easily over the patient 15. The hand piece 11 simply directs radiation from the optical cable 13 to the skin of the patient 15. If required, the hand piece 11 may be provided with an electrical switch means to control the operation of the radiation source.

Cellular Basis for Collagen Formation:

In normal skin an approximate balance exists between collagen formation and degradation. With ageing the degradation rate exceeds that of the formation rate and this process is enhanced by the effects of sun damage. Certain materials such as steroids can have a more drastic effect in reducing the formation rate to near zero. Fibroblasts are responsible for forming pro-collagen (the key component in the construction of collagen molecules) and the activity of the mitochondria control the rate.

Cellular Mechanism of Stimulation:

There exist within the mitochondria a number of cytochromes which have absorption spectra in the visible spectrum.

This present invention exploits the presence of cytochromes in the mitochondria having absorption spectra in the visible spectrum by exciting the mitochondria via absorption of appropriate wavelength radiation (for example between 500 and 800 nm) by one or more of the cytochromes. Although the Applicants do not wish to be bound by any theorem, it is postulated that the resulting energy release, possibly via the release of oxygen, stimulates the mitochondria into an increase in energy production which is the trigger for pro-collagen formation. An additional mechanism might be available through a mechanical interaction, namely the disaggregation of inactive aggregated cytochromes. Thus the invention also provides for irradiation pulse durations which provide for the maximum absorption rate into cytochrome targets and thus a mechanical shock similar to that postulated as leading to the fragmentation of pigment particles in laser removal of tattoos. However, other steps and processes may well be involved. Nevertheless, the above mentioned theorem represents a plausible explanation of the practical demonstration of the process as described further below having regard to the following non-limiting example:

### EXAMPLE

An experiment was conducted to verify the process of light enhanced pro-collagen formation employing light having a wavelength of between 500 and 800 nm. A suction blister technique was employed whereby fluids associated with the dermis were drawn into a collectable zone resembling a blister. This interstitial fluid was then drawn off and analysed by the PIIINP method (Bjerring, P. et al Journ. Cut. Laser Therapy, 2000 (2) pp 9-15) to give an accurate measure of type III pro-collagen production rate. Experiments were conducted with various irradiation parameters using a copper bromide laser at a wavelength of 578 nm. These were compared with non irradiated control zones. The results indicated that the laser irradiation provided a statistically significant increase (circa 140%) in pro-collagen formation.

The irradiation employed in the method of the invention may be provided by a light source offering wavelengths in the visible or near infra-red spectral regions. Such radiation may be provided by an incoherent light source suitably filtered to limit its wavelengths of emission to those absorbed by the target species or by a laser providing an output in a similar spectral region. Such a laser source could for example be a copper bromide laser at 578 nm as described above since this wavelength corresponds to the absorption region of certain cytochromes within the mitochondria. A suitable hand-piece can be used to bring the radiation to the required site; such a hand-piece may be designed to contact the skin and thus enhance the amount of light coupled through to the dermis (as taught by Mills, T. N. and Henderson, A. R. Phys. Med. Biol. 1987 Vol 32 pp 1627-1630).

In another embodiment of the invention a suitable means can be provided for scanning the irradiating beam in a controlled fashion over the desired area of the skin thus providing for more control over the required irradiation density.

The total optical energy density delivered to the skin should be within the range required to initiate simulation whilst remaining below that which might cause undesired damage. Typically this is from 0.5 J/cm² to 30 J/cm² and preferably between 2 and 15 J/cm².

As indicated above, specifically enhancing the photomechanical component of the postulated interaction the source can be pulsed to provide a quasi-continuous stream of short duration pulses each in themselves of low energy. Preferably the duration of such pulses should be between 1 ns and 1 ms, more preferably between 1 ns and 500 ns.

Notwithstanding the pulse duration, in a further embodiment of the invention a pulse shape having a fast-leading edge can be employed. Such a fast leading edge could enhance the mechanical disruption caused to the absorbing target.

In the method of this invention a laser or light source is used to enhance the reformation of collagen via the stimulation of the mitochondria. The dermal layer is irradiated by the light at a suitable wavelength or wavelengths such that chromophores within the mitochondria absorb the radiation preferentially thus sparing the epidermis. The resulting chain of cellular events leads to favourable alterations in the skin geometry, that is for example, a reduction in the size and depth of visible wrinkles. The method of the invention is non-invasive and does not result in or rely on skin ablation or burning.

The invention is not limited to the embodiments herein described which may be varied in construction and detail.

1. A method of stimulating collagen formation in a selected area of mammalian skin comprising the step of irradiating the selected area with a source of visible or near infra-red radiation wherein the radiation is absorbable by intracellular chromophores to enhance cellular activity and increase collagen formation.

2. A method of reducing skin imperfections such as wrinkles, rough textures and other blemishes from a selected area of mammalian skin, comprising the step of irradiating the selected area with a source of visible or near infra-red radiation wherein the radiation is absorbable by intracellular chromophores to enhance cellular activity and increase collagen formation.

3. A method as claimed in claim 1 in which the radiation selected is absorbable by mitochondrial cytochromes.

4. A method according to claim 1 in which the radiation has a wavelength of between 500 and 800 nm.

5. A method according to claim 1 wherein the radiation is provided by an incoherent source suitably filtered to provide the visible or near infra-red radiation.

6. A method according to claim 5 wherein the radiation is provided by a laser source.

7. A method according to claim 6 wherein the laser comprises a copper bromide laser.

8. A method according to claim 1 wherein the delivered energy density of the radiation to the skin is between 0.5 and 30 J/cm².

9. A method according to claim 8 wherein the energy density is between 2 and 15 J/cm².

10. A method according to claim 1 wherein the radiation source comprises a pulsed output.

11. A method according to claim 10 wherein a duration of the pulsed output is between 1 ns and 1 ms.

12. A method according to claim 11 wherein the duration of the pulsed output is between 1 ns and 500 ns.

13. A method according to claim 10 wherein each pulse comprises a sharp leading edge.

14. A method according to claim 4 wherein the radiation has a wavelength of about 578 nm.

15. Use of visible or near infra-red radiation absorbable by intracellular chromophores in the treatment of mammalian skin to reduce skin imperfections such as wrinkles, rough textures or other blemishes from a selected area.

16. A skin treatment device for stimulating collagen formation in mammalian skin comprising a visible or near infra-red radiation source absorbable by intracellular chromophores and an applicator communicable with the radiation source for applying the radiation to mammalian skin.

17. A device as claimed in claim 16 in which the applicator is communicable with the radiation source by means of a fibre or articulating optical device.

18. A device as claimed in claim 16 in which the applicator comprises a hand piece.

19. A device as claimed in claim 16 in which the applicator comprises a scanning device for the controlled irradiation of an enlarged skin area.
